(19) United States
(12) Patent Application Publication
(10) Pub. No.: US 2012/0172590 A1
(43) Pub. Date: Jul. 5, 2012

(54) METHODS FOR ISOLATING ALKALOIDS FROM PLANTS

(76) Inventor: Thomas Kallimopoulos, Ludwigshafen (DE)

(21) Appl. No.: 13/394,432

(22) PCT Filed: Sep. 3, 2010
(86) PCT No.: PCT/EP2010/005433

§ 371 Date: Mar. 6, 2012

(30) Foreign Application Priority Data
Sep. 7, 2009 (DE) 10 2009 040 381.7

(51) Int. Cl. C07D 491/06; C07D 491/147; C07D 453/04

(52) U.S. Cl. 540/581; 546/134; 540/576

(57) ABSTRACT

Methods are provided for isolating alkaloids from biomaterial, preferably plant biomaterial, wherein the biomaterial is extracted with a vegetable oil, in the concomitant presence of an alkaline aqueous phase.

### CROSS-REFERENCE TO RELATED APPLICATIONS

This application is a Section 371/Continuation of International Application No. PCT/EP2010/005433, filed Sep. 3, 2010, which was published in the English language on Mar. 10, 2011, under International Publication No. WO 2011/026637 A2 and the disclosure of which is incorporated herein by reference.

### BACKGROUND OF THE INVENTION

The present invention concerns methods for isolating alkaloids from biomaterial, in particular from plant species. The present invention further comprises the alkaloids that were isolated by the method of the present invention and the use of these alkaloids for manufacturing medicaments.

Alkaloids are naturally occurring, mostly heterocyclic chemical compounds containing at least one basic nitrogen atom. The term “basic nitrogen atom” denotes that this nitrogen atom exhibits basic reactions at neutral pH values. The name “alkaloid” is derived from the word “alkaline” and was used to describe any nitrogen-containing base.

Many alkaloids possess pharmacological activity in mammalian organisms including humans. Examples of pharmacologically useful alkaloids are galanthamine, (−)cephalotaxine and quinine.

Galanthamine (CAS No. 357-70-0; IUPAC name: (4aS,6R,8aS)-5,6,9,10,11,12-hexahydro-3-methoxy-11-methyl-4aH-[1]benzofuro[3a,3,2-ef]-[2]-benzazepin-6-ol) is a cholinesterase inhibitor. Thus, galanthamine can enhance cholinergic function by increasing the concentration of acetylcholine in the central nervous system. Galanthamine has also shown activity in modulating nicotinic cholinergic receptors to increase acetylcholine release.

Galanthamine is used for or proposed to have utility in the treatment of various diseases and disorders as, for example, narrow angle glaucoma, poliomyelitis, Alzheimer's Disease, and various disorders of the nervous system, such as neuropathic pain, alcohol abuse, and smoking cessation. Galanthamine is also used as antidote in organophosphorous poisoning.

Galanthamine is a tetracyclic alkaloid which is mainly present in plants of the genus Amaryllidaceae. Methods for isolating galanthamine from natural source were described, for example, in German published patent application DE 195 09 663 A1. Chemical synthesis of galanthamine has also been described (Kametani et al., Chem. Soc. C., 6:1043-1047 (1971); Shimizu et al., Heterocycles, 8:277-282 (1977)).

(−)Cephalotaxine (CAS No. 24316-19-6; IUPAC name: 1S,3aR,14b)-1,5,6,8,9,14b-Hexahydro-2-methoxy-4H-cyclopenta[a][1,3]dioxolo[4,5-h]pyrrolo[2,1-b][3]benzazepin-1-ol) is the major alkaloid of coniferous bush species of the genus Cephalotaxus, commonly known as Plum Yew or Cowtail Pine. The uniquely structured cephalotaxine itself has no particular anti-tumor activity, but its (alpha)-hydroxysuccinate esters, also known as harringtonins, are inhibitors of angiogenesis and protein biosynthesis, and are promising substances for treating myeloid leukemias. For example, omacetaxine mepesuccinate, a semisynthetic formulation of homoharringtonin, which as formerly known as Ceflatonin® is currently in phase II/III clinical trials for treating chronic myeloid leukemia.

Extraction of cephalotaxine and its subsequent modification remains the method of choice for obtaining sufficient amounts in industrial scale, although several methods for synthesizing racemic mixtures of cephalotaxine have been described since the 1960s, and in 1995 the first synthesis of the pharmaceutically relevant (−)stereoisomer was reported.

In still another example for pharmaceutically active alkaloids, the bark of Cinchona officinalis, C. succirubra, C. ledgeriana and other species of this genus remain the most prominent source of quinine alkaloids. Quinine itself (CAS No. 130-95-0; IUPAC name: (R)-(6-methoxyquinolin-4-yl)((2S,4S,8R)-8-vinylquinuclidin-2-yl)methanol) has regained some of its importance as an anti-malarial agent, and new uses to treat leg cramps and other muscular spasms were discovered recently. Quinidine (CAS No. 56-54-2; IUPAC name: (9S)-6′-methoxycinchonan-9-ol) is a stereoisomer of quinine and is a class I anti-arrhythmic agent, which is used to treat arrhythmias of heartbeats. It is estimated that 300 to 500 metric tons of quinine alkaloids are extracted annually from 5,000 to 10,000 metric tons of Cinchona bark.

Alkaloids are produced by a large variety of organisms, including bacteria, fungi, plants, and animal, as so-called secondary metabolites. Crude plant extracts containing alkaloids were among the first medicines empirically used by mankind From early on, methods for isolating pharmaceutically active alkaloids from alkaloid-containing extracts have attracted interest of pharmacists. To obtain a virtually unlimited supply of specific pharmaceutically active alkaloids at reduced costs, processes for chemical synthesis of these alkaloids have been developed.

For some pharmaceutically active alkaloids, their chemical synthesis has totally supplanted their extraction from natural sources. In the case of some alkaloids, the alkaloid from natural sources and its synthetically produced analogues compete with each other on the global market. For instance, the Johnson & Johnson Group companies use synthetic galanthamine as well as galanthamine extracted from plants to meet its demands for manufacturing Reminyl™ and Razadyne™, both of which are medicaments for treating Alzheimer's disease. In still other cases of medically interesting alkaloids, no process for their chemical synthesis is available that can be employed in an industrial scale at economically competitive conditions.

It is apparent that extraction processes for isolating pharmaceutically active alkaloids from natural sources, in particular from plant species, constitute major processes for obtaining said alkaloids. Ideally, the extraction process itself should already be as selective for the desired alkaloid as possible, while maintaining an acceptable yield of the alkaloid. However, most of the extraction processes for isolating alkaloids utilize a limited number of generic methods, which can be adapted to a relatively broad range of alkaloids to be isolated and to various biomaterials the alkaloid shall be extracted from.

These generic methods rely on the physicochemical properties which alkaloids have in common with each other, but which are absent from non-alkaloid compounds commonly present in the biomaterial containing alkaloids. The most significant physicochemical properties of alkaloids are:
(i) the considerable alteration of the compound's solubility and partition coefficients as the pH value of the solvent changes, which is caused by the presence of one or more basic nitrogen atoms in the molecule; and
(ii) the relatively high polarity conferred by the aromatic ring system and the heteroatoms present within the molecule.

The known generic methods of extracting alkaloids were adapted to and optimized for obtaining particular alkaloids from specific biomaterial of particular botanical species. Most of the methods for isolating alkaloids from natural sources employ extraction of alkaloid-containing material by a polar organic solvent and subsequent acid/base extraction. Instead of an organic polar solvent, supercritical carbon dioxide has been utilized. If the desired alkaloid is known to be glycosylated, undergoes saponification, or possesses other types of chemical modifications which are typical for the plant's secondary metabolism, process steps that specifically target carbohydrate residues or similar moieties can also be included into the extraction process. However, many pharmaceutically active alkaloids are not chemically modified, such that affinity-driven process steps can not be employed. Therefore, isolation of chemically non-modified alkaloids has to rely on the sole extraction with polar solvents and their pH-driven modification of solubility properties.

For example, methods for isolating galanthamine were for instance disclosed in International patent application publication Nos. WO 96/29332 A1, WO 2006/064105 A1 and WO 2006/099635 A1.

German published patent application DE 1 193 061 A1 discloses a method for isolating galanthaminium hydrobromide from members of the genus Amaryllidaceae, wherein air dried and comminuted plant material is alkalized with aqueous ammonia and extracted with dichloroethane. The primary extract is treated with diluted sulphuric acid and accompanying alkaloids are removed from the solution by precipitation with aqueous ammonia. Galanthamine remains in the solution and is further extracted with diethylether or dichloromethane.

WO 96/29332 A1 teaches a method for isolating galanthamine, wherein air-dried, comminuted bulbs of a Narcissus species are mixed with powdered sodium carbonate prior to the first extraction step. The alkalized biomaterial is then extracted with dichloroethane, and the primary extract is further processed as described in DE 1 193 061 A.

In a second example, WO 96/29332 A1 discloses an extraction of the alkalized plant material with special boiling point gasoline as non-halogenated organic solvent to obtain the primary extract. The dried residue of the primary extract is dissolved in diluted sulphuric acid, wherein the pH is adjusted to about 4, and accompanying organic non-alkaloid compounds are removed by extraction with diethyl ether. The refined aqueous solution alkalized to pH 9 and the alkaloids are extracted into diethyl ether.

WO 2006/064105 A1 concerns the use of centrifugal partition chromatography in displacement mode for purifying galanthamine from a starting composition containing at least 20% galanthamine. The method comprises a step of centrifuging a combination of at least two solvents and the starting composition. The two solvents are selected such that they form two non-miscible phases, an aqueous phase and an organic phase.

The starting composition is obtained in that alkalized plant material is extracted with ethylene acetate. The primary extract is treated with diluted sulphuric acid, and accompanying alkaloids are removed from the solution by precipitation with aqueous ammonia. Galanthamine remains in the solution and is further extracted with chloroform.

WO 2006/099635 A1 discloses a process for large scale isolation of galanthamine, wherein the plant material is primarily extracted with an aqueous solution of a suitable organic or inorganic acid. Organic compounds of the thus obtained primary extract are adsorbed on an absorbent, the absorbent is washed with water, and the organic compounds are eluted from the adsorbent using a water miscible organic solvent, such that a concentrate of alkaloids is obtained.

The major drawback of known processes for isolating alkaloids from biomaterial, in particular for isolating galanthamine from plant biomaterial, is the lack of robustness and the lack of scalability for large scale isolation. In addition, the known processes were usually tailor made for a specific source of biomaterial. A given process for a given biomaterial does not enable isolation of sufficient yields of the alkaloid if another biomaterial is used.

Moreover, the use of chlorinated hydrocarbons in most of the known processes discourages these processes for large scale isolation of galanthamine, because of their toxicity and environmental harmfulness. The use of gasoline instead of chlorinated hydrocarbons is inefficient and requires large volumes of this solvent. Furthermore, processes wherein a primary extract has to be dried to dryness and the residue be dissolved in another solvent are difficult to scale up.

### BRIEF SUMMARY OF THE INVENTION

For these reasons, a generic process for isolating alkaloids from natural sources, in particular from plants, is needed that can be modified and optimized for particular configurations of alkaloids and source material, and that can be scaled up to industrial batch sizes.

Surprisingly, it has been found that alkaloids can be efficiently isolated from biomaterial, if a non-volatile and chemically non-modified vegetable oil is used a as solvent in the initial extraction of alkalized plant material.

The extraction process of the present invention comprises the steps of contacting the biomaterial with a vegetable oil or a mixture of vegetable oils and a concomitantly present alkaline aqueous phase to achieve the transfer of the alkaloids from the biomaterial to the oil phase.

The biomaterial is not limiting the extraction process of the present invention, provided that the biomaterial contains the alkaloid that shall be extracted. In case of biomaterial from plants, the plant biomaterial is not limiting the extraction process of the present invention. All parts and tissues of a plant can be employed in the extraction process of the present invention.

In preferred embodiments of the extraction process of the present invention, specific parts or tissues of plants are used, which may be selected from subterranean parts or aerial parts of the plants. Examples of subterranean parts and tissues of plants are roots, rhizomes, tubers and bulbs. Examples of aerial parts or tissues of plants are stems, bark, leafs, buds, flowers, fruits, seeds and galls. It is also possible to employ liquid or semi-liquid contents that are present in any of the plant parts or tissues mentioned. These liquid or semi-liquid contents comprise sap, juices and exudates.

In an embodiment of the extraction process of the present invention, the biomaterial is dried biomaterial. In case of plant biomaterial, the plants, parts of the plants or tissues of the plants to be employed are dried prior to their use in the extraction process. Preferably, the plant material is dried by air-drying or freeze-drying. Air-drying of the plant material can be performed under vacuum or at ambient air pressure, and at ambient temperature or at elevated temperatures.

However, it is not necessary to employ dried biomaterial for performing the extraction process of the present invention. Thus, in a preferred embodiment of the extraction process of the present invention, fresh tissue or fresh parts of plants are used for extracting their alkaloids.

It is preferred that the biomaterial, in particular plant biomaterial, be comminuted prior to the extraction, i.e., the biomaterial is mashed, cut, broken, coarsed, milled, ground, pulverised, or comminuted by any other suitable means.

In the extraction process of the present invention a vegetable oil is employed as a solvent for the initial extraction of the biomaterial.

With respect to the present invention, the term “vegetable oil” refers to any material from a plant that is liquid at room temperature (approx. 23° C.) and composed of triglycerols, free fatty acids, monoglycerols, and diglycerols.

To be a suitable solvent in the extraction process of the present invention, the vegetable oil does not require any chemical modification. Thus straight vegetable oils are preferred vegetable oils in the extraction process of the present invention.

Examples of vegetable oils that are suitable to be used as a solvent in the extraction of alkaloids from biomaterial are rapeseed oil, sunflower oil, linseed oil, grape seed oil, peanut oil, castor oil, pumpkin seed oil, soy bean oil, safflower oil, cotton seed oil, coconut oil, corn oil, castor oil, palm oil, hempseed oil, rice bran oil, tung oil, jojoba oil, and olive oil.

In general, any vegetable oil may be used, regardless of its origin or grade. Although industrial grade vegetable oils may be employed, it is preferred that the vegetable oil be of food grade, veterinary grade or cosmetic grade. The most preferred vegetable oils for the extraction of alkaloids from plant material are edible vegetable oil.

In a preferred embodiment of the process of the present invention, at least 0.1 weight units, preferably at least 0.2 weight units, more preferably at least 0.5 weight units, and most preferably at least 0.8 weight units of vegetable oil are added to each weight unit of biomaterial to be extracted. Preferably, up to 2.0 weight units, preferably up to 3.0 weight units, more preferably up to 5.0 weight units, and most preferably up to 10.0 weight units of vegetable oil are added to each weight unit of biomaterial to be extracted.

In the extraction process of the present invention, the extraction of biomaterial using vegetable oil is performed in the concomitant presence of an alkaline aqueous phase. The alkaline aqueous phase can be an aqueous solution of ammonia, also known as ammonium hydroxide (NH₄OH). In another embodiment, the alkaline aqueous phase is an aqueous solution of an alkali metal carbonate, preferably an aqueous solution of sodium carbonate (Na₂CO₃) or potassium carbonate (K₂CO₃). In another embodiment, the alkaline aqueous phase is an aqueous solution of an alkali metal hydrogen carbonate, preferably an aqueous solution of sodium hydrogen carbonate (NaHCO₃) or potassium hydrogen carbonate (KHCO₃). In still another embodiment, the alkaline aqueous phase is an aqueous solution of an alkali metal hydroxide, preferably an aqueous solution of sodium hydroxide (NaOH) or potassium hydroxide (KOH).

In one embodiment of the present invention, the extraction of the alkaloid or mixture of alkaloids from the biomaterial with the vegetable oil, in concomitant presence of an alkaline aqueous phase, is carried out at ambient temperature. Ambient temperature means room temperature, i.e., the temperature of the air surrounding the extraction vessel which is in the range of between 15° C. and 35° C.

If the extraction is performed at ambient temperature, it is preferred that the biomaterial be contacted with the vegetable oil for a period of between 15 and 30 hours.

In another embodiment, the extraction of the alkaloid or mixture of alkaloids from the biomaterial with the vegetable oil, in concomitant presence of an alkaline aqueous phase, is carried out at elevated temperature, i.e., at a temperature above ambient temperature. In a preferred embodiment, the elevated temperature is in the range of 45° C. to 50° C.

If the extraction is performed at elevated temperature, it is preferred that the biomaterial be contacted with the vegetable oil for a period of between 10 and 60 minutes.

The primary extract obtained from contacting biomaterial with a vegetable oil, in the concomitant presence of an alkaline aqueous phase, is an emulsion which separates in an upper oil phase and a lower aqueous phase. The upper oil phase containing the alkaloid is acidified, for instance by addition of a diluted acid such as sulphuric acid. Preferably, the pH value of the acidified aqueous phase is about pH 2. Upon acidification, the alkaloids are transferred from the oil phase into the acidic aqueous phase.

The acidic aqueous phase is recovered and alkalized, preferably with aqueous ammonia. The preferred pH value of the alkalized aqueous phase is about pH 11. Then, the aqueous phase is extracted with an organic solvent that is immiscible with the aqueous phase. The organic phase is recovered, dried, and the organic solvent is evaporated to obtain a dry residue which contains the alkaloid(s).

The extraction process of the present invention is robust, can be scaled up for large scale isolation of alkaloids from biomass, and significantly reduces the amounts of organic solvents, especially of chlorinated hydrocarbons, compared to known extraction methods.

The extraction process of the present invention can be adapted for isolating a variety of alkaloids, in particular of heterocyclic alkaloids, provided that the hetereocyclic alkaloid is not present as a glycoside or saponin.

The present invention thus extends to the alkaloids that were isolated from biomaterial, preferably from plant material, by the method of the present invention.

The present invention further extends to the use of the alkaloids that were isolated by the method of the present invention for manufacturing a medicament. For example, galanthamine that is isolated by a method of the present invention can be used for manufacturing a medicament for treating narrow angle glaucoma, poliomyelitis, Alzheimer's Disease, and various disorders of the nervous system, such as neuropathic pain, alcohol abuse, smoking cessation, or for preventing organophosphorous poisoning. (−)Cephalotaxine that is isolated by a method of the present invention can be used for manufacturing a medicament for treating chronic myeloid leukemia. Quinine alkaloids that are isolated by a method of the present invention can be used for manufacturing a medicament for treating leg cramps, other muscular spasms, malaria, or arrhythmias of heartbeats.

### DETAILED DESCRIPTION OF THE INVENTION

The present invention will be described herein below with reference to certain specific embodiments thereof. The skilled artisan will understand that the examples are illustrative only and do not construe the invention to any of the specific embodiments described. Those skilled in the art will appreciate that various adaptations, changes and modifications, substitutions, deletions, or additions of procedures and protocols may be made without departing from the merits and the scope of the present invention.

### Example 1

### Extraction of Galanthamine at Ambient Temperature

In a 6-liter vessel, a sodium carbonate solution was prepared in that 0.3 kg granulated Na₂CO₃ was completely dissolved in 1 liter of water.

Two and a half kilograms of clean fresh bulbs of Narcissus cultivar Carlton were chopped and added to the sodium carbonate solution together with 2.2 kg edible rapeseed oil (2,240 ml “Bonita” rapeseed oil from a superstore). The resulting mixture was stirred for 3 min. and then left at ambient temperature (about 23° C.) for 23 hour while being stirred occasionally.

A brownish mass was obtained which was transferred to a Hydrapress balloon fruit press, and the subsequent extrusion yielded approximately 3 kg of an emulsion, which upon rapid decanting separated into an upper oil phase weighing 2,165 g, and a lower dark brown aqueous phase weighing 810 g.

The oil phase was mixed twice with 300 ml of 3.3% H₂SO₄ (pH 2). While the oil phase (1,881 g) was kept as solvent to be used in further extractions, the two recovered acidic aqueous phases (weighing 570 g) were combined.

The combined acidic aqueous phases were extracted once with a mixture consisting of 200 ml cyclohexane and 45 ml NH₄OH (25%) at pH 11. The resulting emulsion was broken by adding two times 2 ml methanol. The organic phase was recovered, dried over MgSO₄, and the solvent was evaporated under vacuum. A yield of 0.441 g of crude galanthamine with a purity of approximately 49% was obtained.

Purity of the extracted galanthamine was determined by HPLC, and identity of galanthamine was confirmed by mass spectroscopy.

### Example 2

### Rapid Extraction of Galanthamine Employing Mild Heat

One kilogram of dried and ground (<5 mm) bulbs from Narcissus cultivar Carlton were added to 1 liter of 10% aqueous ammonia in a 6-liter vessel. While stirred, 1.4 kg rapeseed oil was added, and stirring was continued until the mixture had a homogeneous appearance. The mixture was heated to 40-45° C. in a water bath, and the temperature was maintained over a period of 25 min. Thereafter, the mixture was transferred to a balloon press, and the emulsion was extruded.

After spontaneous phase separation, 1.2 liters of oil phase were recovered and first extracted with 400 ml 3.3% H₂SO₄ and then with 200 ml 3.3% H₂SO₄. The acidic aqueous phases were combined and extracted twice, each time using a mixture of 200 ml cyclohexane and 90 ml 25% aqueous ammonia at pH 11. The organic phases were recovered, dried over MgSO₄, the solvent was removed under vacuum, and 0.473 g galanthamine was obtained.

Purity of the extracted galanthamine was determined by HPLC, and identity of galanthamine was confirmed by mass spectroscopy.

### Example 3

### Extraction of Cephalotaxine

Thirty-six grams Na₂CO₃ were dissolved in 84 ml water in a 100 ml extractor. Forty grams finely mashed fresh leaves of Cephalotaxus harringtonia var. Fastigiata (obtained from Arnold garden services, 56154 Boppard, Germany) and 120 gram rapeseed oil were added to the sodium carbonate solution, which was then mixed thoroughly. The mixture was left standing at ambient temperature (approximately 23° C.) for 21 hours and occasionally stirred.

The solid components were removed by filtration and discarded. The oil phase was separated from the filtered emulsion and extracted with 100 ml 3% H₂SO₄ at pH 2. The aqueous phase was recovered after its separation, and extracted with 87 ml of a 1:1.33 (v/v) mixture of 30% aqueous Na₂CO₃ and CH₂Cl₂ at pH 11. The organic phase was separated, dried of MgSO₄, and the solvent was evaporated, such that 0.020 g of crude cephalotaxine with a purity of 26% was obtained.

Purity of the extracted cephalotaxine was determined by HPLC, and identity of cephalotaxine was confirmed by mass spectroscopy.

### Example 4

### Extraction of Quinine Alkaloids from Cinchona Bark

Fifty-seven grams Na₂CO₃ were dissolved in 192 ml water in a 500 ml extractor. One hundred grams of ground (<0.5 mm) Cinchona bark and 150 g rapeseed oil were added. The mixture was thoroughly mixed and left to stand at ambient temperature (approx. 25° C.) for 19 hours. The mixture was stirred occasionally.

The solid compounds in the mixture were removed by filtration and discarded. The oil phase was separated from the filtered emulsion and extracted with 300 ml 3% H₂SO₄ at pH 2. The aqueous phase was recovered after its separation and washed with 50 ml cyclohexane. Then, the aqueous phase was extracted with 125 ml of a 1:4 (v/v) mixture of 25% aqueous NH₄OH and cyclohexane at pH 11. The resulting emulsion was broken by addition of 5 ml methanol. The organic phase was separated, dried over MgSO₄, and the solvent was evaporated to obtain 0.145 g of a mixture of cinchona alkaloids. The content of quinine in this mixture of cinchona alkaloids was 47%.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1.-14. (canceled)

15. A method for extracting alkaloids from biomaterial, the method comprising the steps of:
comminuting the biomaterial;
contacting the biomaterial with a vegetable oil in the concomitant presence of an alkaline aqueous phase; and
isolating the alkaloid;
wherein the biomaterial is a plant biomaterial.

16. The method according to claim 15, wherein the vegetable oil is selected from the group consisting of rapeseed oil, sunflower oil, linseed oil, grape seed oil, peanut oil, castor oil, pumpkin seed oil, soy bean oil, safflower oil, cotton seed oil, coconut oil, corn oil, castor oil, palm oil, hempseed oil, rice bran oil, tung oil, jojoba oil, and olive oil.

17. The method according to claim 15, wherein the vegetable oil is a plant seed oil and/or edible oil.

18. The method according to claim 15, wherein at least 0.1 weight units of vegetable oil are added to each weight unit of biomaterial to be extracted.

19. The method according to claim 18, wherein at least 0.2 weight units of vegetable oil are added to each weight unit of biomaterial to be extracted.

20. The method according to claim 19, wherein at least 0.5 weight units of vegetable oil are added to each weight unit of biomaterial to be extracted.

21. The method according to claim 20, wherein at least 0.8 weight units of vegetable oil are added to each weight unit of biomaterial to be extracted.

22. The method according to claim 15, wherein up to 2.0 weight units of vegetable oil are added to each weight unit of biomaterial to be extracted.

23. The method according to claim 22, wherein up to 3.0 weight units of vegetable oil are added to each weight unit of biomaterial to be extracted.

24. The method according to claim 23, wherein up to 5.0 weight units of vegetable oil are added to each weight unit of biomaterial to be extracted.

25. The method according to claim 24, wherein up to 10.0 weight units of vegetable oil are added to each weight unit of biomaterial to be extracted.

26. The method according to claim 15, wherein the alkaline aqueous phase is an aqueous solution of an alkali metal carbonate, alkali metal hydrogen carbonate, alkali metal hydroxide, or ammonium hydroxide.

27. The method according to claim 15, wherein the alkaline aqueous phase is selected from the group consisting of aqueous solutions of sodium carbonate, potassium carbonate, sodium hydrogen carbonate, potassium hydrogen carbonate, sodium hydroxide, and potassium hydroxide.

28. The method according to claim 15, wherein the biomaterial is contacted with the vegetable oil at an ambient temperature of between 15° C. and 35° C.

29. The method according to claim 28, wherein the biomaterial is contacted with the vegetable oil for a period of between 15 and 30 hours.

30. The method according to claim 15, wherein the biomaterial is contacted with the vegetable oil at a temperature of between 45° C. and 50° C.

31. The method according to claim 30, wherein the biomaterial is contacted with the vegetable oil for a period of between 10 to 60 minutes.

32. An alkaloid obtained by the method according to claim 15.

33. A medicament comprising an alkaloid according to claim 32.
